(19) United States
(12) Patent Application Publication
(10) Pub. No.: US 2022/0148709 A1
(43) Pub. Date: May 12, 2022

(54) SUPPORTER TERMINAL, SERVER DEVICE, TREATMENT SUPPORT SYSTEM, TREATMENT SUPPORT METHOD, AND PROGRAM

(71) Applicant: LIFE QUEST Inc., Tokyo (JP)

(72) Inventors: Ryozo SAITO, Tokyo (JP); Tetsu KAYAMA, Tokyo (JP); Takuma FUKUDA, Tokyo (JP); Hirotsugu TAKAHASHI, Tokyo (JP); Yuji GAMO, Tokyo (JP); Emi ITO, Tokyo (JP)

(73) Assignee: LIFE QUEST Inc., Tokyo (JP)

(21) Appl. No.: 17/594,683

(22) PCT Filed: Apr. 28, 2019
(86) PCT No.: PCT/JP2019/018172

§ 371 Date: Oct. 26, 2021

(51) Int. Cl. G16H 20/70; G16H 40/67; G16H 80/00

(52) U.S. Cl. CPC G16H 20/70; G16H 80/00; G16H 40/67

(57) ABSTRACT

Deterioration of symptoms of a patient is prevented. A supporter terminal (3-n) contains a display unit (32) for selectably displaying pre-prepared messages effective for treatment of the patient when an event that the patient uses a patient terminal to start training for the treatment is notified through a network, and a terminal control unit (34) for displaying a message selected by a supporter for the patient from among the messages displayed on the display unit (32) on the patient terminal through the network as a message from the supporter.

### TECHNICAL FIELD

The present invention relates to supporter terminals, server devices, treatment support systems, treatment support methods and programs, and in particular to a supporter terminal, a server device, a treatment support system, a treatment support method and a program which can prevent deterioration of a patient's symptom.

### BACKGROUND ART

As a group therapy for children with a developmental disorder such as autism spectrum syndrome and their parental guardians, a program called “parent training” developed in the U.S. in the 1960s has been widely known and has achieved a certain degree of success. Through this “parent training”, the parental guardians can acquire knowledge on how to contact the children with the developmental disorder, how to provide feedback and the like (for example, see patent document 1). In this regard, the specification, the claims and the drawings of the patent document 1 are fully incorporated herein by reference.

### PRIOR ART DOCUMENT

### Patent Document

Patent document 1: JP 2005-215019A

### SUMMARY OF THE INVENTION

### Problem to be Solved by the Invention

Such a group therapy is considered to be also effective for a patient with mild cognitive impairment (Mild Cognitive Impairment; MCIs) or dementia. However, since the patient with the MCI may have symptoms such as memory impairment and disorientation or unstable emotional expressions such as depression and agitation, there is a risk that the patient may become emotional or be deeply injured by mistaking meaning of a supporter's message, and thereby the patient may lose motivation with respect to the training and the symptoms of the patient with the MCI may be deteriorated on the contrary. As described above, it is not easy to smoothly provide the support for the patient with the MCI or the dementia from a surrounding supporter, and thus feedback from the surrounding supporter to the patient with the MCI or the dementia requires careful attention and appropriate knowledge.

The present invention has been made to solve the above-described problem and it is an object of the present invention to provide a supporter terminal, a server device, a treatment support system, a treatment support method and a program which can prevent the deterioration of the patient's symptom.

### Means for Solving the Problem

In order to achieve the above-described object, a supporter terminal (3-n) according to a first aspect of the present disclosure comprises: a display unit (32) for selectably displaying pre-prepared messages effective for treatment of a patient when an event that the patient uses a patient terminal (2) to start training for the treatment is notified through a network (N); and a terminal control unit (34) for displaying a message selected by a supporter for the patient from among the messages displayed on the display unit (32) on the patient terminal (2) through the network (N) as a message from the supporter.

In the above-described supporter terminal (3-n), it is preferable that the display unit (32) selectably displays the messages corresponding to characteristics of the patient.

In the above-described supporter terminal (3-n), the display unit (32) may selectably display the messages in response to an event that the supporter indicates participation in support of the training of the patient after start of the training of the patient is notified.

In the above-described supporter terminal (3-n), it is preferable that the display unit (32) displays a state of the training of the patient.

A server device (4) according to a second aspect of the present invention is connected to a patient terminal (2) to be used by a patient for training for treatment and a supporter terminal (3-n) of a supporter for the patient through a network (N) and comprises: a message database (422) for registering messages effective for the treatment of the patient; and a server control unit (43) for selectably displaying the messages registered in the message database (422) on the supporter terminal (3-n) through the network (N) in response to an event that start of the training of the patient is notified from the patient terminal (2) through the network (N) and displaying the message selected on the supporter terminal (3-n) by the supporter on the patient terminal (2) through the network (N) as a message from the supporter.

It is preferable that the server device (4) further comprises a patient database (421) for registering characteristics of the patient in association with the patient for each patient, the message database (422) registers the messages in association with the characteristics of the patient, and the server control unit (43) reads out the characteristics of the patient from the patient database (421) in response to the event that the start of the training of the patient is notified from the patient terminal (2) through the network (N), detects the messages corresponding to the characteristics of the patient from the message database (422) and selectably displays the messages on the supporter terminal (3-n) through the network (N).

The server device (4) may further comprise a patient database (421) for registering supporter information which can identify the supporter terminal (3-n) of the supporter for the patient in association with the patient for each patient, and the server control unit (43) may read out the supporter information of the patient from the patient database (421) in response to the event that the start of the training of the patient is notified from the patient terminal (2) through the network (N) and selectably display the messages registered in the message database (422) on the supporter terminal (3-n) identified from the supporter information read out from the patient database (421) through the network (N).

In the above-described server device (4), the server control unit (43) may further notify the start of the training of the patient to the supporter terminal (3-n) of the supporter for the patient through the network (N) in response to the event that the start of the training of the patient is notified from the patient terminal (2) through the network (N) and may selectably display the messages registered in the message database (422) on the supporter terminal (3-n) indicating participation in support of the training of the patient through the network (N) in response to an event that the participation in the support of the training of the patient is indicated from the supporter terminal (3-n) through the network (N).

A treatment support system (1) according to a third aspect of the present invention comprises: a server device (4) connected to a patient terminal (2) to be used by a patient for training for treatment through a network; and a supporter terminal (3-n) of a supporter for the patient, the supporter terminal (3-n) connected to the server device (4) through the network (N), wherein the server device (4) includes a message database (422) for registering messages effective for the treatment of the patient, a server control unit (43) for transmitting display data for selectably displaying the messages registered in the message database (422) to the supporter terminal (3-n) through the network (N) in response to an event that start of the training of the patient is notified from the patient terminal (2) through the network (N) and displaying the message identified from a message selection command on the patient terminal (2) through the network (N) as a message from the supporter in response to an event that the message selection command transmitted from the supporter terminal (3-n) through the network (N) is received, and wherein the supporter terminal (3-n) includes a display unit (32) for selectably displaying the messages registered in the message database (422) based on the display data in response to an event that the display data transmitted from the server device (4) through the network (N) is received, and a terminal control unit (34) for transmitting a message selection command indicating a message selected by the supporter from among the messages displayed on the display unit (32) to the server device (4) through the network (N).

A treatment support method according to a fourth aspect of the present invention is characterized in that the treatment support method comprises: selectably displaying pre-prepared messages effective for treatment of a patient on a display unit (32) when an event that the patient uses a patient terminal (2) to start training for the treatment is notified through a network (N); and displaying a message selected by a supporter for the patient from among the messages displayed on the display unit (32) on the patient terminal (2) through the network (N) as a message from the supporter.

A treatment support method according to a fifth aspect of the present invention is a treatment support method provided by a server device (4) connected to a patient terminal (2) to be used by a patient for training for treatment and a supporter terminal (3-n) of a supporter for the patient through a network (N) and including a message database (422) for registering messages effective for the treatment of the patient and the treatment support method is characterized in that: the treatment support method comprises: selectably displaying the messages registered in the message database (422) on the supporter terminal (3-n) through the network (N) in response to an event that start of the training of the patient is notified from the patient terminal (2) through the network (N); and displaying the message selected on the supporter terminal (3-n) by the supporter on the patient terminal (2) through the network (N) as a message from the supporter.

A treatment support method according to a sixth aspect of the present invention is a treatment support method provided by a treatment support system (1) containing a server device (4) connected to a patient terminal (2) to be used by a patient for training of treatment through a network and including a message database (422) for registering messages effective for the treatment of the patient and a supporter terminal (3-n) of a supporter for the patient, the supporter terminal (3-n) connected to the server device (4) through the network (N) and the treatment support method is characterized in that: the server device (4) transmits display data for selectably displaying the messages registered in the message database (422) to the supporter terminal (3-n) through the network (N) in response to an event that start of the training of the patient is notified from the patient terminal (2) through the network (N), the supporter terminal (3-n) selectably displays the messages effective for the treatment of the patient the display unit (32) based on the display data in response to an event that the display data transmitted from the server device (4) through the network (N) is received, the supporter terminal (3-n) transmits a message selection command indicating a message selected by the supporter from among the messages displayed on the display unit (32) to the server device (4) through the network (N), and the server device (4) displays the message identified from the message selection command on the patient terminal (2) through the network (N) as a message from the supporter in response to an event that the message selection command transmitted from the supporter terminal (3-n) through the network (N) is received.

A program according to a seventh aspect of the present invention causes a computer to execute a procedure comprising: selectably displaying pre-prepared messages effective for treatment of a patient on a display unit (32) when an event that the patient uses a patient terminal (2) to start training for the treatment is notified through a network (N); and displaying a message selected by a supporter for the patient from among the messages displayed on the display unit (32) on the patient terminal (2) through the network (N) as a message from the supporter.

A program according to a eighth aspect of the present invention causes a computer of a server device (4) connected to a patient terminal (2) to be used by a patient for training for treatment and a supporter terminal (3-n) of a supporter for the patient through a network (N) and including a message database (422) for registering messages effective for the treatment of the patient to execute a procedure comprising: selectably displaying the messages registered in the message database (422) on the supporter terminal (3-n) through the network (N) in response to an event that start of the training of the patient is notified from the patient terminal (2) through the network (N); and displaying the message selected on the supporter terminal (3-n) by the supporter on the patient terminal (2) through the network (N) as a message from the supporter.

### Effect of the Invention

According to the present invention, it is possible to provide a supporter terminal, a server device, a treatment support system, a treatment support method and a program which can prevent deterioration of a patient's symptoms.

### BRIEF DESCRIPTION OF THE DRAWINGS

FIG. 1 is a diagram showing a configuration example of a treatment support system according to the present embodiment.

FIG. 2 is a block diagram showing a configuration example of a patient terminal.

FIG. 3 is a diagram showing a display example of a play screen.

FIG. 4 is a block diagram showing a configuration example of a supporter terminal.

FIG. 5 is a diagram showing a display example of a message selection screen.

FIG. 6 is a block diagram showing a configuration example of a server device.

FIG. 7 is a flowchart showing details of a play start notification process.

FIG. 8 is a flowchart showing details of a treatment support process.

FIG. 9 is a flowchart showing a continuation of the treatment support process.

### MODE FOR CARRYING OUT THE INVENTION

Hereinafter, description will be given to a best mode for carrying out the present invention.

First, a configuration of a treatment support system according to an embodiment of the present invention will be described with reference to the accompanying drawings.

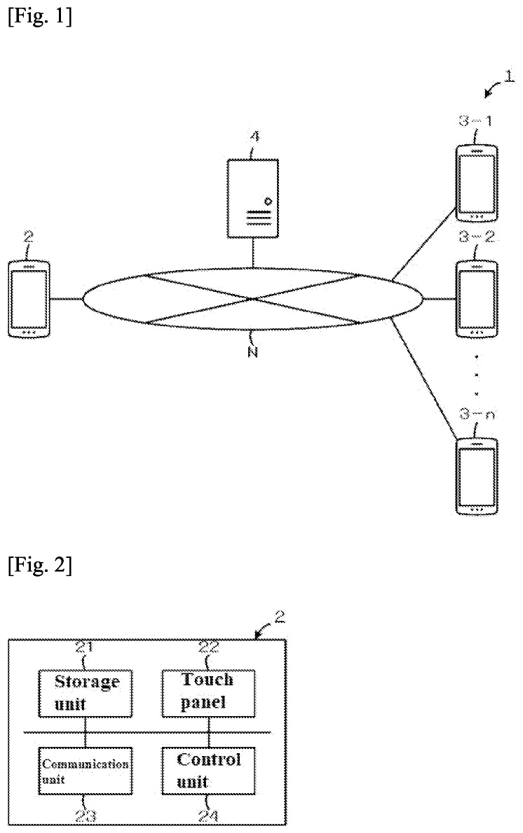

FIG. 1 is a diagram showing a configuration example of the treatment support system according to the present embodiment.

As shown in FIG. 1, a treatment support system 1 includes a patient terminal 2, supporter terminals 3-n (“n” is a natural number) and a server device 4, which are communicably connected to each other through a network N such as the Internet.

The patient terminal 2 is composed of, for example, a general-purpose smart phone, a tablet computer, a personal computer or the like. The patient terminal 2 is used by, for example, a patient with mild cognitive impairment (Mild Cognitive Impairment; MCI) for training for treatment.

FIG. 2 is a block diagram showing a configuration example of the patient terminal.

As shown in FIG. 2, the patient terminal 2 includes a storage unit 21, a touch panel 22, a communication unit 23 and a control unit 24, which are connected to each other through a bus or the like.

The storage unit 21 is composed of, for example, a non-volatile memory or the like such as a general-purpose flash memory. Various application programs such as an application program for treating the patient with the MCI (hereinafter referred to as a “treatment program”) are installed in the storage unit 21. The treatment program contains various games or the like for providing training of a brain of the patient to prevent development of the MCI.

The touch panel 22 is composed of, for example, a general-purpose touch panel consisted by combining a liquid crystal display device and a pointing device or the like. The touch panel 22 displays various screens as well as accepts various operations from the patient. In the present embodiment, the patient plays the various games and the like contained in the treatment program on a play screen displayed on the touch panel 22.

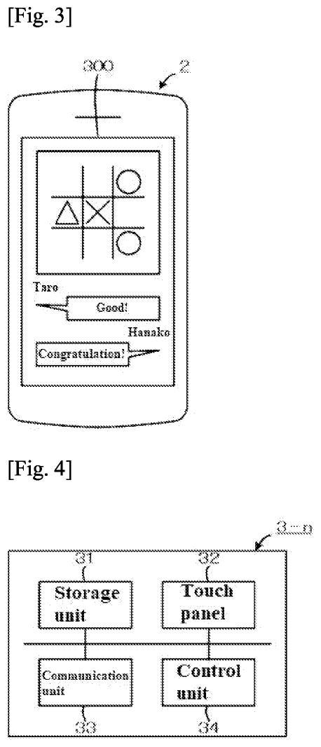

FIG. 3 is a diagram showing a display example of the play screen.

As shown in FIG. 3, while the game being played by the patient is playing is displayed on a play screen 300, a display name of a surrounding supporter such as a patient's family and relative who is closely related to the patient and a message (feedback message) such as “Good!” and “Congratulations!” from the supporter who cheers the patient playing the game are displayed below the game.

The communication unit 23 shown in FIG. 2 is composed of, for example, a general-purpose wireless communication device or the like. The communication unit 23 transmits a first play start notification notifying an event that the patient starts to play the game to the server device 4 through the network N to the server device 4. The communication unit 23 receives a message display command transmitted from the server device 4 through the network N.

The control unit 24 is composed of, for example, a CPU (Central Processing Unit), a ROM (Read Only Memory), a RAM (Random Access Memory) or the like. The CPU uses the RAM as a work memory and appropriately executes various programs or the like stored in the ROM and the storage unit 21 to control various operations of the patient terminal 2.

In the present embodiment, the control unit 24 activates the treatment program stored in the storage unit 21 in response to an event that the patient taps an icon indicating the treatment program displayed on the touch panel 22 and displays a top screen of the treatment program on the touch panel 22. Next, the control unit 24 displays the play screen 300 for playing the game on the touch panel 22 in response to an event that the patient selects the game to be played on the top screen. In addition, the control unit 24 transmits the first play start notification containing a patient identifier which can identify the patient who starts the game to the server device 4 from the communication unit 23 through the network N.

The control unit 24 displays the display name of the supporter identified from the message display command and a message selected by this supporter below the game being played by the patient on the play screen 300 in response to an event that the message display command transmitted from the server device 4 through the network N is received at the communication unit 23.

The supporter terminal 3-n shown in FIG. 1 is composed of a general-purpose smart phone, a tablet computer, a personal computer or the like, similarly to the patient terminal 2. The supporter terminal 3-n is carried by the surrounding supporter such as the patient's families, the relative or the like who is closely related to the patient.

FIG. 4 is a block diagram showing a configuration example of the supporter terminal.

As shown in FIG. 4, the supporter terminal 3-n includes a storage unit 31, a touch panel 32, a communication unit 33 and a control unit (terminal control unit) 34, which are connected through a bus or the like.

The storage unit 31 is composed of a non-volatile memory such as a general-purpose flash memory or the like, similar to the storage unit 21. Various application programs such as an application program (hereinafter referred to as “support program”) for supporting the patient playing the game are installed in the storage unit 31.

The touch panel 32 is composed of a general-purpose touch panel or the like, similar to the touch panel 22. The touch panel 32 displays various screens as well as accepts various operations from the supporter. In the present embodiment, a message selection screen for selecting a message for cheering the patient playing the game from among a plurality of kinds is displayed on the touch panel 32.

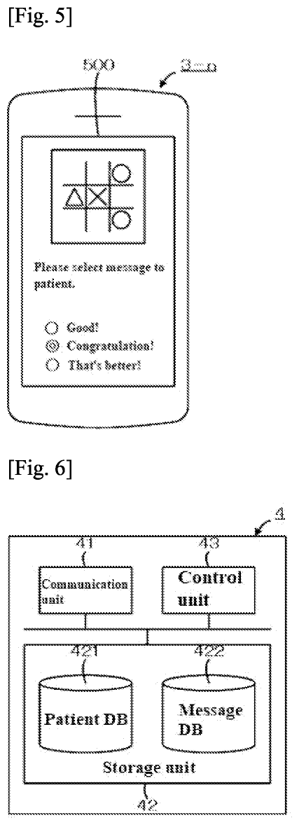

FIG. 5 is a diagram showing a display example of the message selection screen.

As shown in FIG. 5, while messages such as “Good!”, “Congratulations!”, “Good Job!” and the like are selectably displayed on a message selection screen 500, a situation of the game being played by the patient is displayed above the messages. With this configuration, the situation of the game being played by the patient is also shared with the supporter. The supporter taps to select a message cheering the patient playing the game on the message selection screen 500 with taking into account the situation of the game being played by the patient.

The communication unit 33 shown in FIG. 2 is composed of a general-purpose wireless communication device or the like, similarly to the communication unit 23. The communication unit 33 receives a second play start notification transmitted from the server device 4 through the network N, display data for displaying the message selection screen 500 and the like. In addition, the communication unit 33 transmits a cheering participation request requesting participation in cheering for the patient playing the game or a message selection command indicating the message selected by the supporter to the server device 4 through the network N.

The control unit 34 is composed of a CPU, a ROM, a RAM or the like, similar to the control unit 24. The CPU uses the RAM as a work memory and appropriately executes various programs or the like stored in the ROM and the storage unit 31 to control various operations of the supporter terminal 3-n.

In the present embodiment, the control unit 34 notifies the event that the patient starts to play the game to the patient to the supporter by displaying it on the touch panel 32, outputting a sound from a speaker (not shown) or providing a push notification by using a vibrator (not shown) in response an event that the second play start notification transmitted from the server device 4 through the network N is received at the communication unit 33.

Next, while the control unit 34 activates the support program stored in the storage unit 31 in response to an event that the supporter taps an icon indicating the support program displayed on the touch panel 32 to indicate the participation in the cheering for the patient playing the game, the control unit 34 transmits a cheering participation request containing a supporter identifier which can identify the supporter from the communication unit 33 to the server device 4 through the network N.

The control unit 34 displays the message selection screen 500 on the touch panel 32 based on the display data in response to an event that the display data of the message selection screen 500 transmitted from the server device 4 through the network N is received at the communication unit 32. Then, the control unit 34 transmits the message selection command containing the message selected by the supporter and the supporter identifier which can identify this supporter from the communication unit 32 through the network N in response to an event that the supporter taps to select the message on the message selection screen 500.

The server device 4 shown in FIG. 1 is composed of, for example, a general-purpose server computer, a general-purpose database (DataBase; DB) or the like.

FIG. 6 is a block diagram showing a configuration example of the server device.

As shown in FIG. 6, the server device 4 includes a communication unit 41, a storage unit 42 and a control unit (server control unit) 43, which are connected to each other through a bus or the like.

The communication unit 41 is composed of, for example, a NIC (Network Interface Card) or the like. The communication unit 41 receives the first play start notification transmitted from the patient terminal 2 through the network N. The communication unit 41 transmits the second play start notification notifying the event that the patient starts to play the game to the supporter terminal 3-n or the display data of the message selection screen 500 to the supporter terminal 3-n through the network N. Further, the communication unit 41 receives the cheering participation request or the message selection command transmitted from the supporter terminal 3-n through the network N. The communication unit 41 transmits the message display command commanding the patient terminal 2 to display the message selected by the supporter through the network N.

The storage unit 42 is composed of, for example, a general-purpose hard disk drive or the like. In the present embodiment, the storage unit 42 includes a patient DB 421 and a message DB 422.

The patient DB 421 registers, for each patient, information on each patient (hereinafter referred to as “patient information”) and information which can identify the supporter terminal 3-n of the supporter around each patient (hereinafter referred to as “supporter information”) in association with each other. The patient information contains, for example, the patient identifier which can identify the patient, the name and the characteristics (such as gender, age, disease state, personality and the like) of the patient and the like. The supporter information contains, for example, the supporter identifier which can identify the supporter, the display name of the supporter, a relationship with the patient, an e-mail address and the like.

The message DB 422 registers a plurality of kinds of messages from the supporter who cheers the patient playing the games in association with the characteristics of the patient.

For improvement of the patient's symptoms with the MCI, support from the surrounding supporter such as the patient's family and relative who is closely related to the patient is absolutely essential. The message from the supporter cheering the patient playing the game to train the patient's brain is also very useful for the improvement of the patient's symptoms with the MCI because the message can make the patient confident and maintain motivation of the patient with respect to the training. However, since the patient with the MCI may have symptoms such as memory impairment and disorientation or unstable emotional expressions such as depression and agitation, there is a risk that the patient may become emotional or be deeply injured by mistaking meaning of the message of the supporter or the like, and thereby the patient may lose the motivation with respect to the training and the symptoms of the patient with the MCI may be deteriorated on the contrary. Therefore, in the present embodiment, only the messages effective for the treatment of the patient with the MCI are pre-prepared in the message DB 422 and the supporter selects the message cheering the patient playing the game from the message DB 422 to interact with the patient, so that it is possible to prevent a message which may results in the deterioration of the symptoms of the patient with the MCI from being issued.

The messages registered in the message DB 422 are supervised and appropriately updated by a group therapy specialist such as a specialty doctor and a therapist who is appropriately knowledgeable about the treatment of the patient with the MCI.

The control unit 43 is composed of, for example, a CPU, a ROM, a RAM or the like. The CPU uses the RAM as a work memory and appropriately executes various programs or the like stored in the ROM and the storage unit 42 to control various operations of the server device 4.

In the present embodiment, the control unit 43 reads out the supporter information and the patient information corresponding to the patient identifier contained in the first play start notification from the patient DB 421 in response to an event that the first play start notification transmitted from the patient terminal 2 through the network N is received at the communication unit 41. Next, the control unit 43 transmits the second play start notification from the communication unit 41 to the supporter terminal 3-n carried by the supporter identified from the supporter information through the network N.

The control unit 43 detects a plurality of kinds of messages corresponding to the characteristics of the patient contained in the patient information read out from the patient DB 321 from the message DB 422 in response to an event that the cheering participation request transmitted from the supporter terminal 3-n through the network N is received at the communication unit 41. Subsequently, the control unit 43 generates the display data of the message selection screen 500 for selecting the message for cheering the patient playing the game from among the plurality of kinds of messages detected from the message DB 422. Then, the control unit 43 transmits the display data of the message selection screen 500 from the communication unit 41 through the network N to the supporter terminal 3-n carried by the supporter identified from the supporter identifier contained in the cheering participation request through the network N.

The control unit 43 transmits the message display command containing the display name of the supporter identified from the supporter information corresponding to the supporter identifier contained in the message selection command and the message contained in the message selection command to the patient terminal 2 from the communication unit 41 through the network N in response to an event that the message selection command transmitted from the supporter terminal 3-n through the network N is received at the communication unit 41.

Next, various processes executed by the treatment support system 1 including the above-described configuration will be described with reference to the drawings.

The treatment support system 1 starts a play start process on the top screen of the treatment program displayed on the touch panel 22 of the patient terminal 2 in response to the event that the patient selects the game to be played.

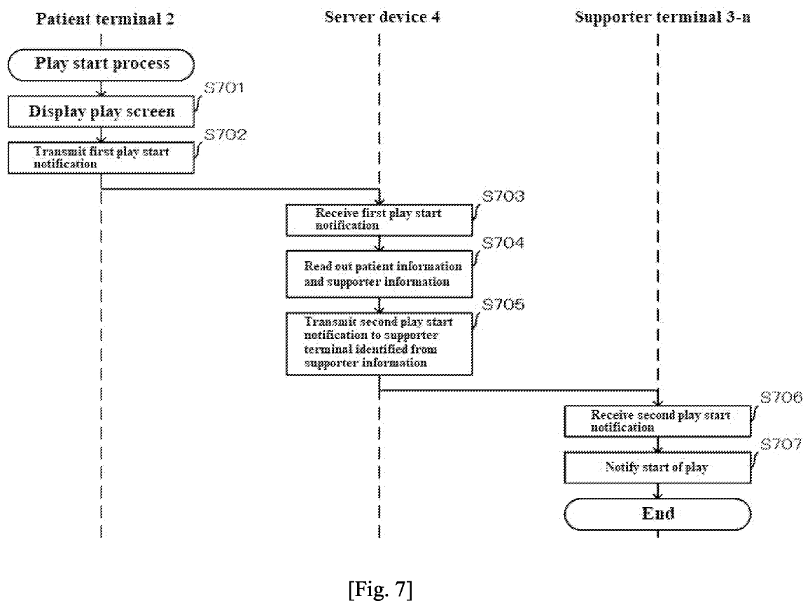

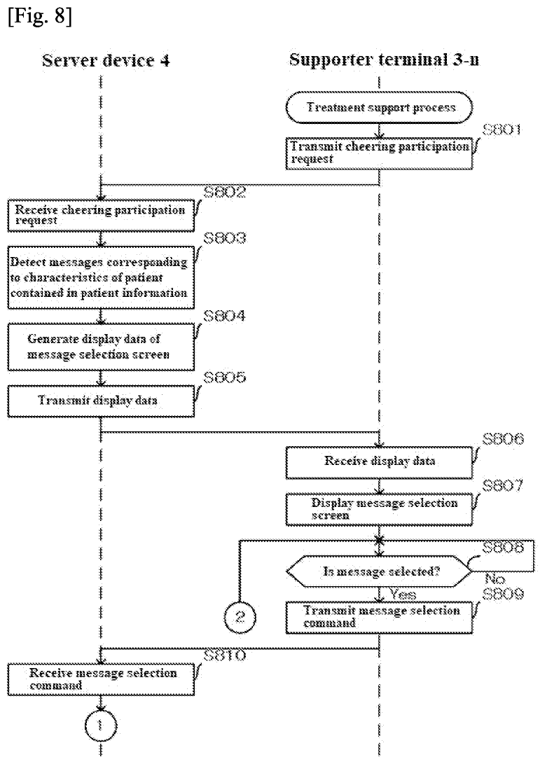

FIG. 7 is a flowchart showing details of the play start process.

In the play start process shown in FIG. 7, the control unit 24 of the patient terminal 2 first displays the play screen 300 for playing the game on the touch panel 22 (step S701).

In addition, the control unit 24 transmits the first play start notification containing the patient identifier which can identify the patient who starts the game from the communication unit 23 to the server device 4 through the network N (step S702).

The control unit 43 of the server device 4 responds to the event that the first play start notification transmitted from the patient terminal 2 through the network N is received at the communication unit 41 (step S703) to read out the supporter information and the patient information corresponding to the patient identifier contained in the first play start notification from the patient DB 421 (step S704).

Next, the control unit 43 transmits the second play start notification from the communication unit 41 to the supporter terminal 3-n carried by the supporter identified from the supporter information through the network N (step S705).

The control unit 34 of the supporter terminal 3-n responds to the event that the second play start notification transmitted from the server device 4 through the network N is received at the communication unit 33 (step S706) to notify the event that the patient starts to play the game to the supporter (step S707) and then terminates the play start process.

Thereafter, in response to the event that the supporter taps the icon indicating the support program displayed on the touch panel 32 to indicate the participation in the cheering for the patient playing the game, the support program stored in the storage unit 31 is activated to start the treatment support process.

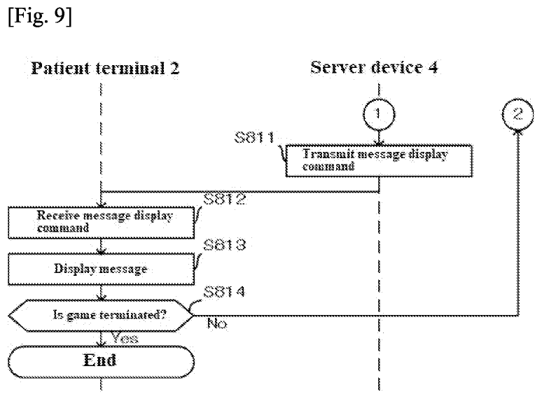

FIG. 8 and FIG. 9 are flowcharts showing details of the treatment support process.

In the treatment support process shown in FIGS. 8 and 9, the control unit 34 first transmits the cheering participation request containing the supporter identifier which can identify the supporter from the communication unit 33 to the server device 4 through the network N (step S801 shown in FIG. 8).

The control unit 43 of the server device 4 responds to the event that the cheering participation request transmitted from the supporter terminal 3-n through the network N is received at the communication unit 41 (step S802) to detect the plurality of kinds of messages, which correspond to the characteristics of the patient contained in the patient information read out from the patient DB 321, from the message DB 422 (step S803).

Subsequently, the control unit 43 generates the display data of the message selection screen 500 for selecting the message for cheering the patient playing the game from among the plurality of kinds of messages detected from the message DB 422 (step S804).

Then, the control unit 43 transmits the display data of the message selection screen 500 from the communication unit 41 through the network N to the supporter terminal 3-n carried by the supporter identified from the supporter identifier contained in the cheering participation request (step S805).

The control unit 34 of the supporter terminal 3-n responds to the event that the display data of the message selection screen 500 transmitted from the server device 4 through the network N is received at the communication unit 32 (step S806) to display the message selection screen 500 on the touch panel 32 based on the display data (step S807).

Then, the control unit 34 responds to the event that the supporter taps to select the message on the message selection screen 500 (step S808; Yes) to transmit the message selection command containing the message selected by the supporter and the supporter identifier which can identify this supporter from the communication unit 32 through the network N (step S809).

The control unit 43 of the server device 4 responds to the event that the message selection command transmitted from the supporter terminal 3-n through the network N is received at the communication unit 41 (step S810) to transmit the message display command containing the display name of the supporter identified from the supporter information corresponding to the supporter identifier contained in the message selection command and the message contained in the message selection command from the communication unit 41 to the patient terminal 2 through the network N (step S811 shown in FIG. 9).

The control unit 24 of the patient terminal 2 responds to the event that the message display command transmitted from the server device 4 through the network N is received at the communication unit 23 (step S812) to display the display name of the supporter identified from the message display command and the message selected by this supporter below the game being played by the patient on the play screen 300 (step S813).

Until the patient finishes the play of the game (step S814; No), the control unit 24 returns to the step S808 and waits for an event that the supporter selects a new message in the message selection screen 500. When the patient finishes the play of the game (step S814; No), the control unit 24 terminates the treatment support process.

As described above, the treatment support system 1 according to the present embodiment includes the patient terminal 2 to be used by the patient for the training for the treatment, the support terminal(s) 3-n of the supporter(s) for the patient and the server device 4 which is connected to these elements through the network N and includes the patient DB 421 and the message DB 422. The patient DB 421 registers, for each patient, the characteristics of the patient and the supporter information which can identify the supporter terminal(s) 3-n of the supporter(s) for the patient in association with each other. The message DB 422 registers the messages effective for the treatment of the patient in association with the characteristics of the patient.

The control unit (server control unit) 43 of the server device 4 reads out the characteristics of the patient and the supporter information from the patient DB 421 in response to the event that the start of the training (the play of the game) of the patient is notified by the first play start notification transmitted from the patient terminal 2 through the network N. Then, the control unit 43 further notifies the start of the training of the patient to the supporter terminal 3-n of the supporter for this patient by transmitting the second play start notification to the supporter terminal identified from the supporter information read out from the patient DB 421 through the network N. The supporter terminal 3-n transmits the cheering participation request to the server device 4 through the network N in response to the event that the supporter indicates the participation in the support of the training of the patient (the cheering for the patient playing the game) after the start of the training of the patient is notified.

Thereafter, the control unit 43 detects the messages, which correspond to the characteristics of the patient read out from the patient DB 421, from the message DB 422 in response to the event that the participation in the support of the training of the patient is indicated by the cheering participation request transmitted from the supporter terminal 3-n through the network N. Then, the control unit 43 transmits the display data for selectably displaying the messages detected from the message DB 422 to the supporter terminal 3-n identified from the supporter information read out from the patient DB 421, thereby selectably displaying the messages effective for the treatment of the patient on the supporter terminal 3-n indicating the participation in the support of the training of the patient.

The supporter terminal 3-n selectably displays the messages corresponding to the characteristics of the patient and effective for the treatment of the patient on the display unit 32 based on the display data in response to the event that the display data transmitted from the server device 4 through the network N is received. Further, the display unit 32 displays the status of the training of the patient. Further, the control unit (terminal control unit) 34 of the supporter terminal 3-n transmits the message selection command indicating the message selected by the supporter from among the messages displayed on the display unit 32 to the server device 4 through the network N.

The control unit 43 of the server device 4 displays the message identified from the message selection command on the patient terminal 2 through the network N as the message from the supporter in response to the event that the message selection command transmitted from the supporter terminal 3-n through the network N is received.

As described above, in the present embodiment, only the messages effective for the treatment of the patient with the MCI are pre-prepared in the message DB 422 under the supervision of the specialist who is appropriately knowledgeable about the treatment of the patient with the MCI and the supporter selects the message cheering the patient playing the game from the message DB 422, and thereby it is possible to autonomically and safely interact with the patient to effectively support the patient. With this configuration, it is possible to prevent the deterioration of the symptoms of the patient with the MCI. Further, it is also possible to find out and address the symptoms of the patient with the MCI at an early stage. In addition, mental and physical health of the supporter is improved by providing a “psychological support” such as reducing stress of the supporter (family and the like) other than the patient himself/herself. As a result, it is also possible to contribute to the improvement of the treatment effect of the patient himself/herself.

In this regard, the present invention is not limited to the above-described embodiment and various modifications and applications are possible. Hereinafter, a modified aspect of the above-described embodiment applicable to the present invention will be described.

In the above-described embodiment, the description has been provided with citing the MCI as an example of the treatment target. However, the present invention is not limited thereto, and the treatment target is arbitrary as long as the patient can perform the training for the treatment by using the patient terminal 2 and may be dementia, developmental disorder, gambling dependency or the like. Alternatively, it may be a disease of an internal medicine such as hypertension, chronic pain, cancer or the like.

In the above-described embodiment, programs executed by the CPUs of the control units 24, 34, 43 have been described as being stored in advance in the ROM, the storage units 21, 31, 42 or the like, but the present invention is not limited thereto, and programs for executing the above-described processes may be applied to an existing general-purpose computer to allow it to serve as the patient terminal 2, the supporter terminal(s) 3-n and the server device 4 according to the above-described embodiment.

A method of providing such a program is arbitrary. For example, the program may be stored in a computer-readable storage medium (a flexible disk, a CD (Compact Disc)-ROM, a DVD (Digital Versatile Disc)-ROM or the like) and distributed, or may be stored in a storage device on a network such as the Internet and provided by downloading it.

Further, if the above-described processes are executed by assignation tasks of an OS (Operating System) and an application program or cooperation tasks of the OS and the application program, only the application program may be stored in a storage medium or a storage device. Further, it is possible to superimpose the program on a carrier wave to distribute the program through a network. For example, the above-described program may be posted to a bulletin board (BBS: Bulletin Board System) on a network to distribute the program through the network. Further, it may be possible to configure so that the above-described processes are executed by activating this program under the control of the OS in the same manner as other application programs.

In this regard, it should be noted that various embodiment and modifications can be applied to the present invention without departing from the spirit and the scope of the present invention in a broad sense. In addition, it should be noted that the above-described aspects are intended to explain one embodiment of the present invention and do not limit the scope of the present invention.

### DESCRIPTION OF REFERENCE SINGS

1 Treatment support system
2 Patient terminal
3-n Supporter terminal
4 Server device
21,31,42 Storage unit
22,32 Touch panel
23,33,41 Communication unit
24,34,43 Control unit
300 Play screen
421 Patient DB
422 Message DB
500 Message selection screen

1-14. (canceled)

15. A supporter terminal (3-n), comprising:
a display unit (32) for selectably displaying pre-prepared messages effective for treatment of a patient in response to an event that a supporter for the patient indicates participation in support of training of the patient after an event that the patient uses a patient terminal (2) to start the training for the treatment is notified through a network (N); and
a terminal control unit (34) for displaying a message selected by the supporter for the patient from among the messages displayed on the display unit (32) on the patient terminal (2) through the network (N) as a message from the supporter.

16. The supporter terminal (3-n) claimed in claim 15, wherein the display unit (32) selectably displays the messages corresponding to characteristics of the patient.

17. The supporter terminal (3-n) claimed in claim 15, wherein the display unit (32) displays a state of the training of the patient.

18. A server device (4) connected to a patient terminal (2) to be used by a patient for training for treatment and a supporter terminal (3-n) of a supporter for the patient through a network (N), the server device (4) comprising:
a message database (422) for registering messages effective for the treatment of the patient; and
a server control unit (43) for further notifying start of the training of the patient to the supporter terminal (3-n) of the supporter for the patient through the network (N) in response to an event that the start of the training of the patient is notified from the patient terminal (2) through the network (N), selectably displaying the messages registered in the message database (422) on the supporter terminal (3-n) indicating participation in support of the training of the patient through the network (N) in response to an event that the participation in the support of the training of the patient is indicated from the supporter terminal (3-n) through the network (N) and displaying the message selected on the supporter terminal (3-n) by the supporter on the patient terminal (2) through the network (N) as a message from the supporter.

19. A treatment support system (1), comprising:
a server device (4) connected to a patient terminal (2) to be used by a patient for training for treatment through a network; and
a supporter terminal (3-n) of a supporter for the patient, the supporter terminal (3-n) connected to the server device (4) through the network (N),
wherein the server device (4) includes:
a message database (422) for registering messages effective for the treatment of the patient, a server control unit (43) for further notifying start of the training of the patient to the supporter terminal (3-n) of the supporter for the patient through the network (N) in response to an event that the start of the training of the patient is notified from the patient terminal (2) through the network (N), transmitting display data for selectably displaying the messages registered in the message database (422) to the supporter terminal (3-n) indicating participation in support of the training of the patient through the network (N) in response to an event that the participation in the support of the training of the patient is indicated from the supporter terminal (3-n) through the network (N) and displaying the message identified from a message selection command on the patient terminal (2) through the network (N) as a message from the supporter in response to an event that the message selection command transmitted from the supporter terminal (3-n) through the network (N) is received, and
wherein the supporter terminal (3-n) includes:
a display unit (32) for selectably displaying the messages effective for the treatment of the patient based on the display data in response to an event that the display data transmitted from the server device (4) through the network (N) is received, and
a terminal control unit (34) for transmitting a message selection command indicating a message selected by the supporter from among the messages displayed on the display unit (32) to the server device (4) through the network (N).

20. A treatment support method, comprising:
selectably displaying pre-prepared messages effective for treatment of a patient on a display unit (32) in response to an event that a supporter for the patient indicates participation in support of training of the patient after an event that the patient uses a patient terminal (2) to start the training for the treatment is notified through a network (N); and
displaying a message selected by the supporter for the patient from among the messages displayed on the display unit (32) on the patient terminal (2) through the network (N) as a message from the supporter.

21. A treatment support method provided by a server device (4) connected to a patient terminal (2) to be used by a patient for training for treatment and a supporter terminal (3-n) of a supporter for the patient through a network (N) and including a message database (422) for registering messages effective for the treatment of the patient, the treatment support method comprising:
further notifying start of the training of the patient to the supporter terminal (3-n) of the supporter for the patient through the network (N) in response to an event that the start of the training of the patient is notified from the patient terminal (2) through the network (N), selectably displaying the messages registered in the message database (422) on the supporter terminal (3-n) indicating participation in support of the training of the patient through the network (N) in response to an event that the participation in the support of the training of the patient is indicated from the supporter terminal (3-n) through the network (N); and
displaying the message selected on the supporter terminal (3-n) by the supporter on the patient terminal (2) through the network (N) as a message from the supporter.

22. A treatment support method provided by a treatment support system (1) containing a server device (4) connected to a patient terminal (2) to be used by a patient for training of treatment through a network and including a message database (422) for registering messages effective for the treatment of the patient and a supporter terminal (3-n) of a supporter for the patient, the supporter terminal (3-n) connected to the server device (4) through the network (N),
wherein the server device (4) further notifies start of the training of the patient to the supporter terminal (3-n) of the supporter for the patient through the network (N) in response to an event that the start of the training of the patient is notified from the patient terminal (2) through the network (N),
wherein the server device (4) transmits display data for selectably displaying the messages registered in the message database (422) to the supporter terminal (3-n) indicating participation in support of the training of the patient through the network (N) in response to an event that the participation in the support of the training of the patient is indicated from the supporter terminal (3-n) through the network (N),
wherein the supporter terminal (3-n) selectably displays the messages registered in the message database (422) on the display unit (32) based on the display data in response to an event that the display data transmitted from the server device (4) through the network (N) is received, wherein the supporter terminal (3-n) transmits a message selection command indicating a message selected by the supporter from among the messages displayed on the display unit (32) to the server device (4) through the network (N), and
wherein the server device (4) displays the message identified from the message selection command on the patient terminal (2) through the network (N) as a message from the supporter in response to an event that the message selection command transmitted from the supporter terminal (3-n) through the network (N) is received.

23. A server device (4) connected to a patient terminal (2) to be used by a patient for training for treatment and a supporter terminal (3-n) of a supporter for the patient through a network (N), the server device (4) comprising:
a message database (422) for registering messages effective for the treatment of the patient; and
a server control unit (43) for selectably displaying the messages registered in the message database (422) on the supporter terminal (3-n) through the network (N) in response to an event that start of the training of the patient is notified from the patient terminal (2) through the network (N) and displaying the message selected on the supporter terminal (3-n) by the supporter on the patient terminal (2) through the network (N) as a message from the supporter, the server device (4) further comprising a patient database (421) for registering characteristics of the patient in association with the patient for each patient,
wherein the message database (422) registers the messages in association with the characteristics of the patient, and
wherein the server control unit (43):
reads out the characteristics of the patient from the patient database (421) in response to the event that the start of the training of the patient is notified from the patient terminal (2) through the network (N), and
detects the messages corresponding to the characteristics of the patient from the message database (422) to selectably display the messages on the supporter terminal (3-n) through the network (N).

24. A server device (4) connected to a patient terminal (2) to be used by a patient for training for treatment and a supporter terminal (3-n) of a supporter for the patient through a network (N), the server device (4) comprising:
a message database (422) for registering messages effective for the treatment of the patient; and
a server control unit (43) for selectably displaying the messages registered in the message database (422) on the supporter terminal (3-n) through the network (N) in response to an event that start of the training of the patient is notified from the patient terminal (2) through the network (N) and displaying the message selected on the supporter terminal (3-n) by the supporter on the patient terminal (2) through the network (N) as a message from the supporter, the server device (4) further comprising a patient database (421) for registering supporter information which can identify the supporter terminal (3-n) of the supporter for the patient in association with the patient for each patient,
wherein the server control unit (43):
reads out the supporter information of the patient from the patient database (421) in response to the event that the start of the training of the patient is notified from the patient terminal (2) through the network (N), and
selectably displays the messages registered in the message database (422) on the supporter terminal (3-n) identified from the supporter information read out from the patient database (421) through the network (N).
